(19) United States
(12) Patent Application Publication
(10) Pub. No.: US 2007/0197945 A1
(43) Pub. Date: Aug. 23, 2007

(54) Hip and pelvic splint

(76) Inventor: Steven T. Islava, Newport Beach, CA (US)

Correspondence Address: LINER YANKELEVITZ SUNSHINE AND REGENSTREIF, 1100 GLENDON AVENUE, 14TH FLOOR, LOS ANGELES, CA, 90024, US

(21) Appl. No.: 11/357,623

(22) Filed: Feb. 17, 2006

(51) Int. Cl. A61F 5/00

(52) U.S. Cl. 602/24

(57) ABSTRACT

A hip and pelvic splint system is designed to immobilize both the pelvic region and lower extremities of a patient suffering from a hip or pelvic fracture. This allows ready transport of the patient with little or no pain or additional injury caused by shifting of the hip or pelvis. The device consists of a foam leg insert that is first placed between the legs of a patient to help stabilize the patient's lower extremities. A flexible splint component is then slid beneath the patient's lets and hips. The splint component is then closed over the patient and tightened with straps. The lower edge of splint component is secured snuggly about the legs in the vicinity of the patient's knees so that the foam leg insert can immobilize the legs. The upper edge of the splint is secured snuggly about the upper abdomen, thereby immobilizing the pelvis. Hand holds on the device allow paramedics to lift the patient without distorting the fracture.

### CROSS-REFERENCE TO RELATED U.S. APPLICATIONS

This application claims priority from Provisional Application U.S. Ser. No. 60/654,026 filed on Feb. 16, 2005, the contents of which are incorporated herein by reference.

### BACKGROUND OF THE INVENTION

1. Area of the Art

The present invention is in the area of medical devices and is more specifically directed to a simple device to provide temporary stabilization of hip and pelvic fractures.

2. Background

Over 300,000 patients in the United States suffer a fracture in the hip region every year. Fractures of the hip and pelvic region are among the most common and devastating injuries—particularly among senior citizens. Older individuals often experience loss of strength, coordination and eye sight. Combined with an increasing fragility of the skeleton serious fractures are a constant danger. Hip and pelvic fractures prove especially troublesome. They are often slow and difficult to recover from. Painful and dangerous surgery may be required. Many seniors suffering these fractures never regain their mobility. This loss of mobility seems to correlate with a rapid downturn in overall health. It is not surprising to have a seemingly healthy senior deteriorate rapidly following a hip fracture. Even if rapid deterioration is not the immediate result, a loss of mobility greatly increases the cost of life for the senior as round the clock help may be required. Thereby putting additional strain on our medical and care giving facilities.

It now appears that initial trauma care immediately following the fracture may strongly influence the overall outcome. Hip and pelvic fractures include not only fractures of the proximal femur but also fracture of the pelvis and acetabulum. Any motion between the torso and lower limbs can cause severe shifting of a fractured pelvis, further damaging tissues and leading to internal bleeding with subsequent coagulation and blood clot formation as well as causing extreme pain to the patient. If the injured patient is further damaged during transport to the nearest hospital, chances for a speedy and complete recovery are diminished and/or slowed considerably.

There have been some attempts to provide an easy to use splint for use with pelvic fractures. Typical are U.S. Pat. Nos. 6,503,217 and 6,793,639 to Gibbs et al. However, the devices disclosed in these patents enclose only the pelvis and fail to immobilize the lower extremities. This allows the possibility for considerable additional injury and pain resulting from shifting of the legs during transport. Therefore, there remains a significant need for an improved device to stabilize hip and pelvic fractures during patient transport.

### SUMMARY OF THE INVENTION

A hip and pelvic splint system is designed to immobilize both the pelvic region and lower extremities of a patient suffering from a hip or pelvic fracture. This immobilization allows ready transport of the patient with little or no pain or additional injury caused by shifting of fractured bones. The device consists of two components: an optional foam leg insert and a sheet-like flexible splint component which has at least two straps. To use the device the patient is laid out on his or her back and, if necessary, the foam leg insert is placed between the legs of the patient to help stabilize the patient's lower extremities. The foam insert is elongate and sized to fit between the upper portions of the patient's legs above the knees. The insert preferably has rounded grooves on either side to receive the legs. After the foam insert is in position, the flexible splint component is then carefully slid beneath the patient's lets and hips. This leaves the patient lying on the sheet-like splint component with the foam insert between the patient's legs. The lower edge of the splint component is below the pelvis in the vicinity of the patient's knees. The upper edge of the splint component is above the pelvis at the upper part of the abdomen or the lower part of the rib cage.

The sheet-like splint component is formed from a sheet resilient material having a corrugated interior. The corrugations reinforce the material and make it deformable in one direction and resistant to deformation at a direction normal to the deformable direction. Thus, it is possible to readily roll up the sheet in a direction normal to the long axis of the corrugations (i.e., the long axis of the resulting roll will be parallel to the long axis of the corrugations). Because the corrugations are relatively rigid, the sheet cannot be rolled or easily deformed in a direction parallel to the long axis of the corrugations. In use the patient is placed parallel to the long axis of the corrugations so that the sheet-like splint component can be easily rolled around the patient.

The splint component is then rolled up and closed over the patient and tightened with integral straps. The lower edge of splint component is secured about the legs near the patient's knees so that the foam leg insert can immobilize the legs. The upper edge is secured about the upper abdomen, thereby immobilizing the pelvis. Hand holds which may be connected to the straps allow paramedics to lift the patient without distorting the fracture. The components of the device are all transparent to x-rays so that x-ray images of the fracture can be produced without having to remove the splint and risk shifting of the bones. The device can be advantageously left on the patient until the fracture is surgically repaired (assuming surgery can be scheduled relatively rapidly). Following surgery the device can be used to immobilize the patient during recovery.

The device can be easily compactly rolled or folded for storage in an impermeable plastic bag or film. Prior to shipment the bag can be evacuated and sealed further reducing the storage size since both the sheet-like splint component and the foam insert are compressed by evacuation. When the storage bag is opened, the components rapidly spring back to their original size. The plastic materials used for the device permit ready sterilization before or after packaging.

### DESCRIPTION OF THE FIGURES

FIG. 1 is of the entire device in an extended configuration;

FIG. 2 is a close-up view of the device of FIG. 1 taken within the circle marked 2;

FIG. 3 is a side view along the plane 3-3 in FIG. 1;

FIG. 4 is close-up view along the plane 4-4 in FIG. 2;

FIG. 5 is a perspective view of the sponge insert shown in FIG. 3;

FIG. 6 is a diagrammatic view if the device of FIG. 1 in relation to a portion of the body of a patient;

FIG. 7 shows the device of FIG. 1 rolled up;

FIG. 8 shows a vacuum collapsed rolled-up device of FIG. 7 enclosed in a gas impermeable protective bag;

FIG. 9 shows a perspective view of the device of FIG. 1 in position beneath a patient ready to enclose the patient for transport;

FIG. 10 shows a perspective view of a first step in enclosing a patient in the device;

FIG. 11 shows a perspective view of a patient enclosed in the device ready for transport;

FIG. 12 shows a view of a patient being transported without a back board;

FIG. 13 is a cross-section view of the rolled-up device taken along plane 13-13 of FIG. 7;

FIG. 14 is a cross-section view of the roll-up collapsed device taken along plane 14-14 in FIG. 8

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide an improved hip and pelvic splint device that minimizes motion of a patient's lower extremities.

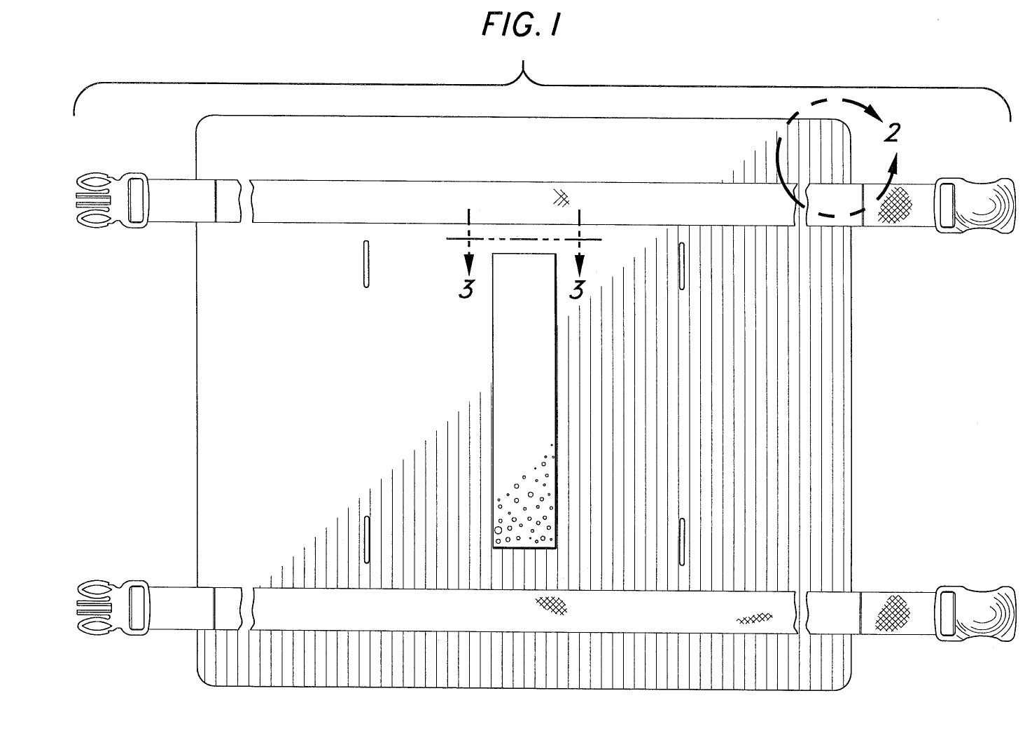

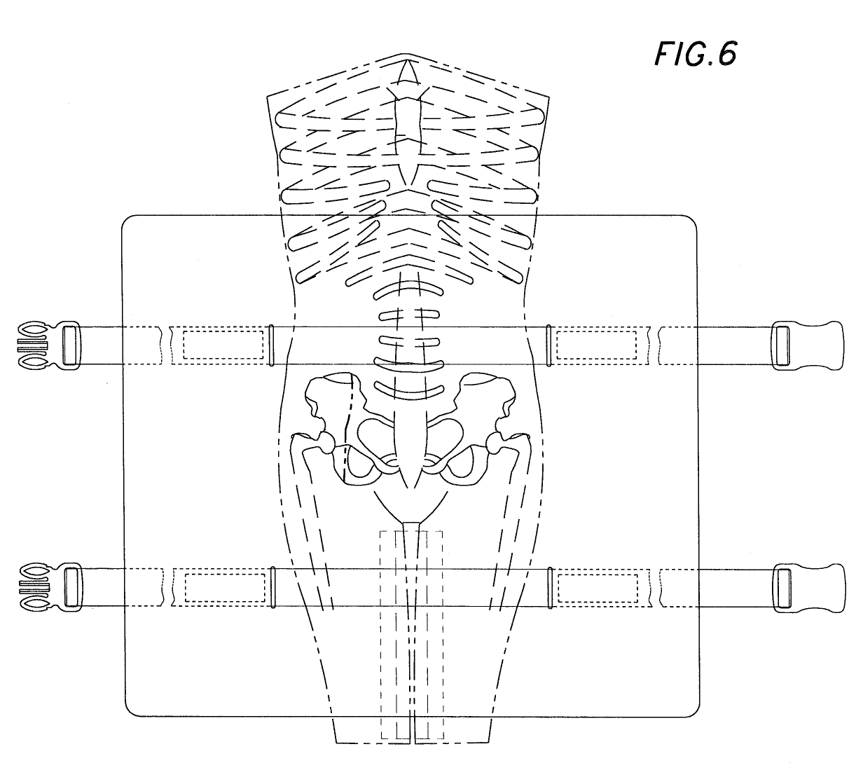

The key to transporting and moving patients with suspected hip fracture is to immobilize the hip and lower extremities to prevent any movement during transport. The inventive Hip and Pelvic splint 20 is designed to quickly stabilize the hip and region with circumvention compression. The splint 20 is designed to control movement of the hip and pelvic region as well as movement of the lower extremities. As shown in FIG. 1 the device 20 consists of a flexible, planar splint component 22 to which are affixed at least two straps 24. One embodiment of the device is about 30 inches by 36 inches. This size is adequate for the great majority of patients. The straps are typical woven webbing of nylon or polyester although other suitable strap material can be use the straps 24 may be adhered to one surface of the splint component, may pass through “belt loops” on the splint component 22 or, as shown in FIG. 6, the straps 24 may be affixed to the splint component 22 by passing through slots 36. When the device 20 is in use one end of a strap 24 is connected to the opposite end of the same strap by buckles 26, 26′ or similar fastening devices; the buckles 26, 26′ allow the strap 24 to be tightened to remove slack. In addition, the straps are preferably equipped with lifting-handle loops 30 to allow emergency medical personnel to lift a patient enclosed by the splint component 22. Alternately, hand holds can be fastened directly to the splint component 22.

Because the device 20 is intended to immobilize the lower extremities as well as the hip and pelvis, the splint component 22 encloses a larger region of a patient's body than most prior art devices. FIG. 6 shows the extended splint component 22 in place underneath the body 32 (shown in “X-ray” view) of a prone patient. Note that the upper edge of the splint component 22 extends above the patient's pelvic region 33. If the splint component 22 interacts with a portion of the patient's upper abdomen near the edge of the rib cage 35, the abdomen can help control the size of the “tube” formed by the rolled splint component 22 so that its circumvention exerts stabilizing pressure but not excessive force on the pelvic region 33 (as might happen with a device cinched or tightened at the pelvis), thereby stabilizing the hip and pelvis. Further, the lower edge 23 of the splint component 22 reaches near (depending on the size of the patient) the knee region of the patient. A foam insert 28 is provided to help stabilize the patent's lower extremities. The foam insert 28 has a girder or beam-like shape (see FIGS. 3 and 5) with flat surfaces 29 and rounded grooves 31 to interact with the splint component 22 and the patient's legs, respectively. If a patient has particularly large or obese legs, the foam insert 28 can be omitted.

If the foam insert 28 is used on an injured patient, the foam insert 28 is first placed between the legs of the patient 32 to stabilize the legs. Then, the extended splint component 22 is gently slid beneath the patient 32 with the straps perpendicular to the patient's body. When the splint component 22 is ultimately rolled and tightened around the patient's legs, the legs are pressed into the rounded grooves 31, and the friction between the legs, the splint component 22 and the foam insert 28 completely, yet gently, immobilizes the lower extremities.

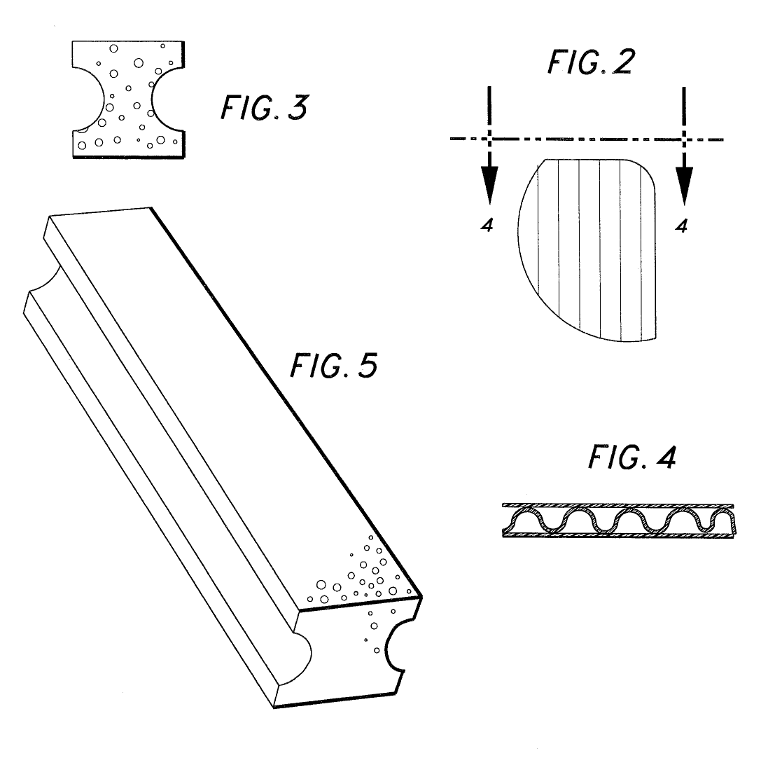

As shown in FIGS. 2 and 4, the splint component 22 is a planar sheet of material having a corrugated core with the long axis of the corrugations 19 running perpendicular to the straps 24 of the splint component 22. The splint component 22 comprises a corrugated material 19 sandwiched between two flat layers similar to the structure of corrugated cardboard. The component 22 can be a traditional cardboard material or can be composed of sheets of durable plastic polymer or of cardboard infiltrated with or encapsulated in a polymer. The corrugations 19 provide the sheet with differential direction properties. Thus, the splint component 22 shows a great deal of flexibility in a “permissible” direction normal to the long axis of the corrugations 19 and can be readily “rolled” about a patient to cocoon or envelop the patient. While the splint component 22 is quite flexible in the permissible direction, it is almost rigid in a direction normal to the permissible direction. Thus, the slat component 22 resist bending in the non-permissible direction and while is can bend resiliently to some degree, it resists molding itself to the patient's exact contour. Thus, while it provides gentle stabilizing pressure to the pelvis while cushioning it.

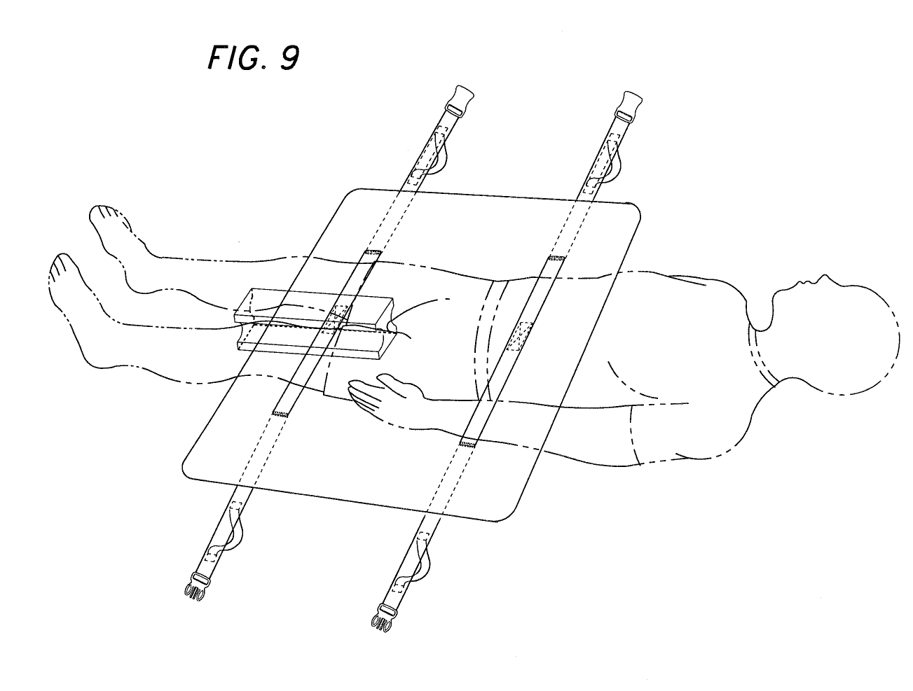

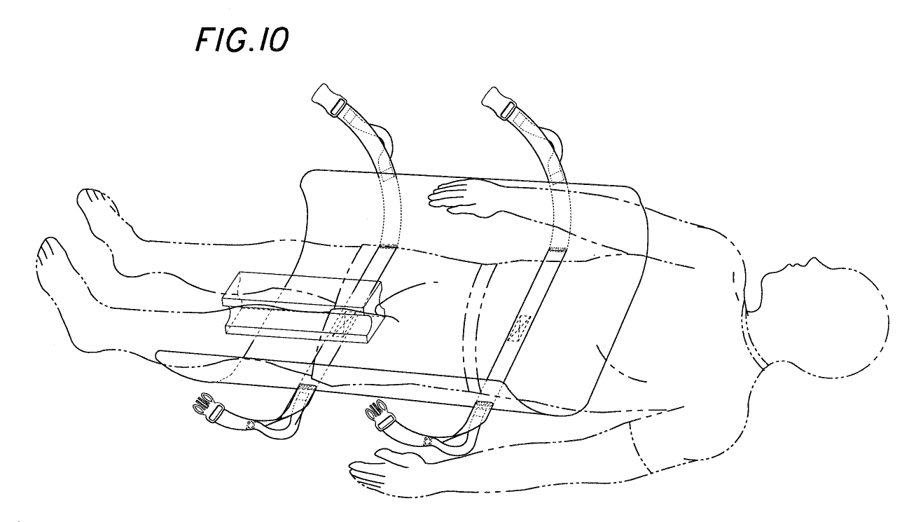

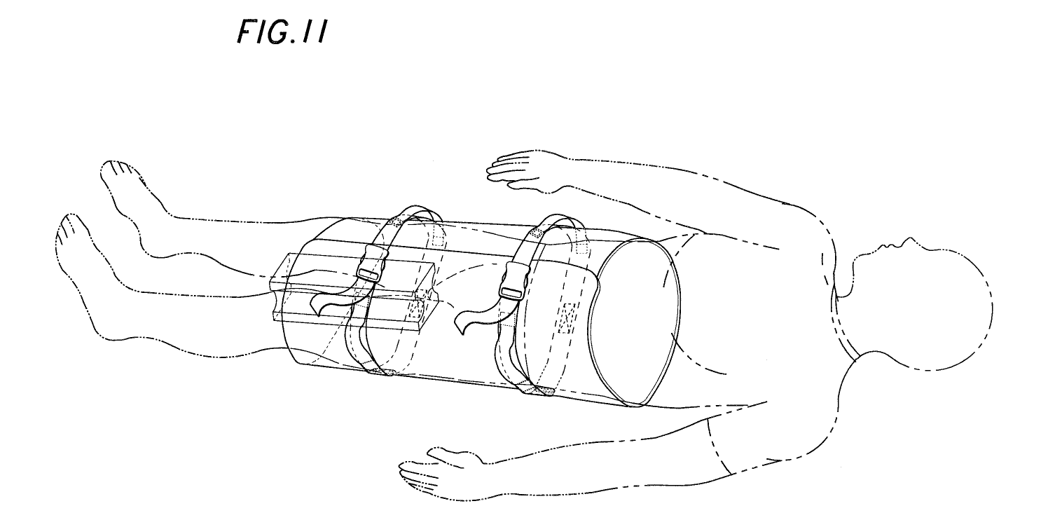

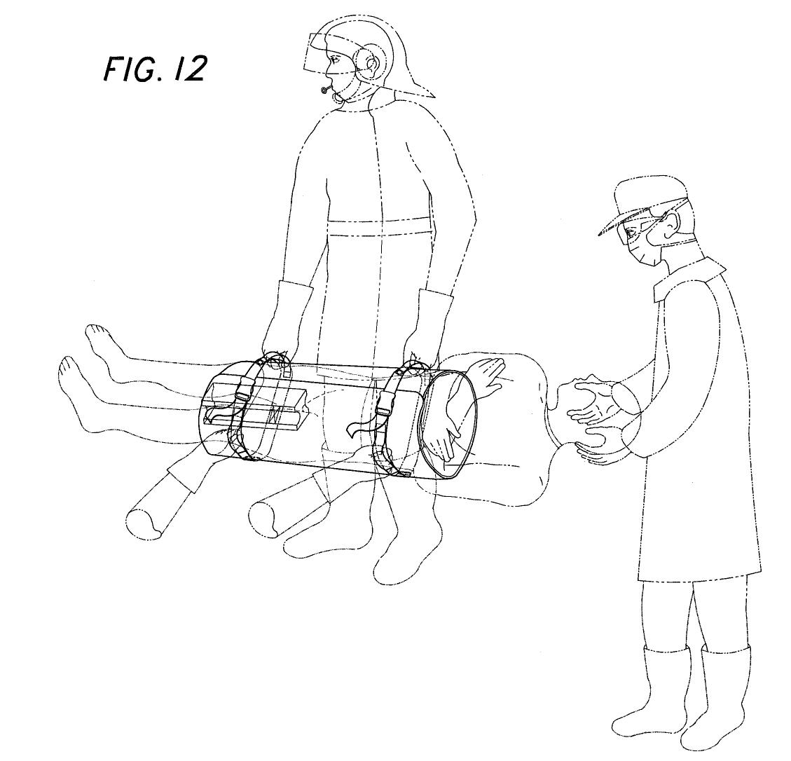

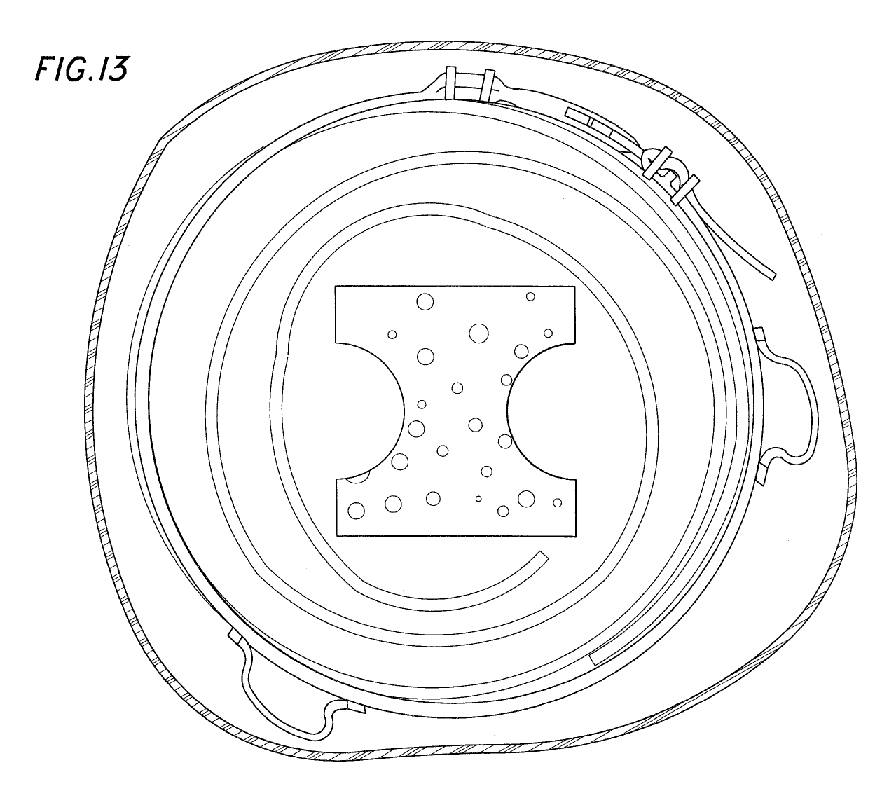

FIGS. 9, 10 and 11 show the successive steps of using the device 20 on a patient 32. In FIG. 9 emergency personnel have already placed the foam inset 28 between the patient's legs (assuming that it is needed with this particular patient) and slid the extended splint component 22 beneath the patient 32. FIG. 10 shows the initial stages of wrapping the splint component 22 around the patient 32. It is most convenient to have one paramedic conform the splint component 22 around the hips as a second paramedic buckles and tightens the straps as shown in FIG. 11. Finally, in FIG. 11 the splint component 22 is shown wrapped snugly about the patient 32 and the straps 24, connected by buckles 26, 26′, have been tightened to remove slack. The lower strap 24 constrains the device 20 snuggly around the patient's lower extremities where the foam insert 28 prevents movement. The upper strap 24 constrains the device 20 snuggly around the patient's middle or upper abdomen. The pelvic region is now enclosed and immobilized by the tube formed by the splint component 22 without any excessive or crushing pressure being applied to the pelvis. FIG. 12 depicts several emergency personnel 38 transporting an enveloped patient. The handle-loop regions 30 on the straps make it relatively simple to lift the patient without applying pressure to the pelvic region. To prevent the straps 24 from slipping when the handle-loop regions are lifted, the straps 24 are preferably attached permanently to the splint component 22 at one or more points by rivets, staples, adhesive or other fastening means. Depending on the situation the patient can be lifted onto a back board or gurney for transport.

The splint device 20 comforts patients by assuring them that pain from movement will be minimized. The patient who is supported and cocooned by the splint 20 will feel safe and secure with little or no movement of the fracture, thereby greatly reducing the pain during patient transport from the accident scene to the gurney followed by ambulance transport to the hospital. Unlike the unstabilized patient who will feel every bump and jostle in the road, additional pain when lifting patient to hospital bed, additional pain on movement to and x-ray tables and additional pain when returned to the hospital bed as well as potential injury due to shifting of fractured bones, the enveloped patient will feel none of this. All portions of the device 20 are transparent to x-rays so that images can be taken without removing the device 20.

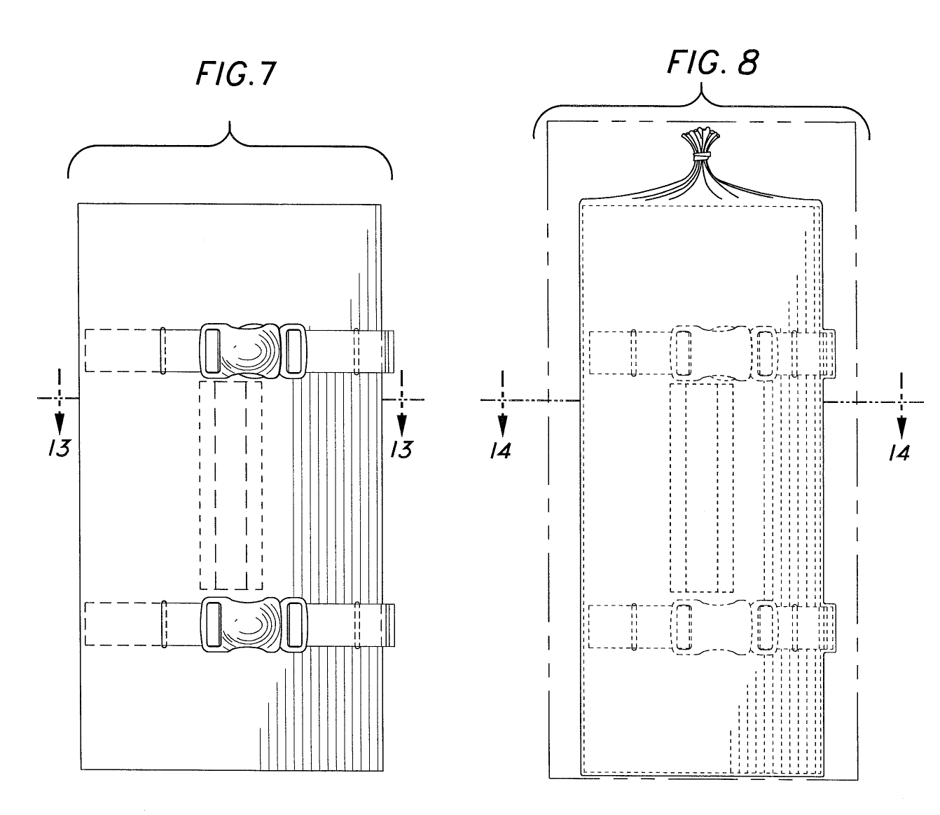

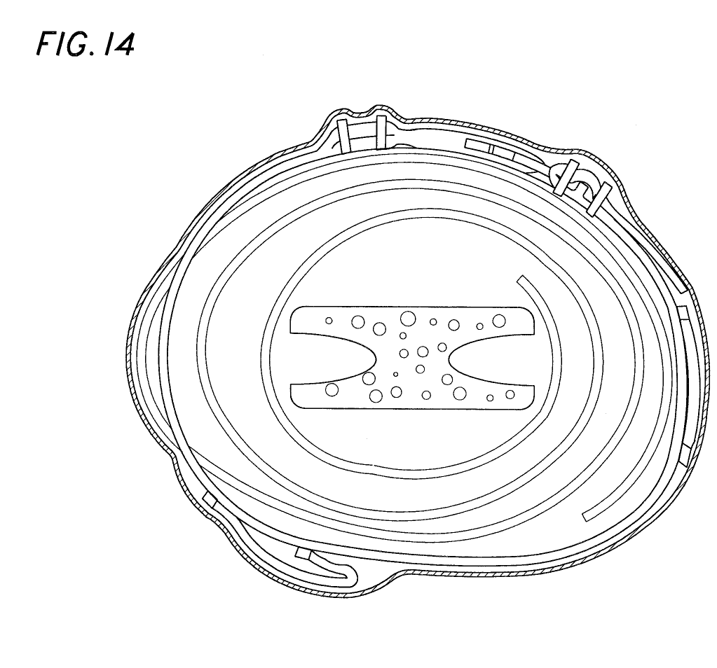

Not only is the splint device 20 easy to use and effective at preventing patient pain and additional injury, it is convenient to store in emergency vehicles. As shown in FIGS. 7 and 8 the splint component 22 can easily be compacted for storage purposes. The splint component can be rolled up light a tight bed roll and after enclosure in an air impermeable bag 34, the unit can be “vacuum packed” to further reduce its size. The bag 34 can be evacuated causing the splint component to be further compacted (compare FIGS. 13 and 14). If the foam insert 28 is constructed from open cell plastic foam, evacuation will cause the insert 28 to decrease greatly in size. Alternatively, the splint component 22 can be folded in a zigzag or accordion fashion (rather like an automobile sunshade) rather than being rolled for storage. In either case after vacuum packing the result is a compact rectangular package only a few inches high allowing a considerable number of units to be stored in a compact space. When the impermeable bag 34 is opened, the foam insert 28 rapidly takes in air and returns to full size.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

1. A hip and pelvic splint system comprising:
a planar sheet of material capable of being rolled up in a first direction and resisting being rolled up in a second direction normal to the first direction; and
a pair of straps running parallel to the first direction and cooperating with the planar sheet to hold the planar sheet in a rolled up configuration.

2. The hip and pelvic splint system according to claim 1, wherein the planar sheet comprises a corrugated material.

3. The hip and pelvic splint system according to claim 1, further comprising a resilient foam insert sized and shaped to fit between the legs of a patient.

4. The hip and pelvic splint system according to claim 3, wherein the resilient foam insert is formed from an open cell plastic foam.

5. The hip and pelvic splint system according to claim 1 further comprising a storage container of air impermeable film which envelops the system.

6. The hip and pelvic splint system according to claim 5, wherein the storage container is evacuated to compress the system.

7. The hip and pelvic splint system according to claim 1, further comprising hand holds to facilitate lifting the system when a patient is enclosed therein.

8. The hip and pelvic splint system according to claim 7, wherein the hand holds are attached to the straps.

9. A hip and pelvic splint system comprising:
a splint component comprising:
a planar sheet of material capable of being rolled up in a first direction and resisting being rolled up in a second direction normal to the first direction; and
a pair of straps running parallel to the first direction and cooperating with the sheet to hold the sheet in a rolled configuration; and
an resilient foam insert sized and shaped to fit between the legs of a patient.

10. The hip and pelvic splint system according to claim 9, wherein the planar sheet comprises a corrugated material.

11. The hip and pelvic splint system according to claim 9, wherein the resilient foam insert is formed from an open cell plastic foam.

12. The hip and pelvic splint system according to claim 9 further comprising a storage container of air impermeable film which envelops the system.

13. The hip and pelvic splint system according to claim 12, wherein the storage container is evacuated to compress the system.

14. The hip and pelvic splint system according to claim 9, further comprising hand holds to facilitate lifting the system when a patient is enclosed therein.

15. The hip and pelvic splint system according to claim 14, wherein the hand holds are attached to the straps.

16. A method for immobilizing a hip or pelvic fracture of a patient to prevent pain and further injury comprising the steps of:
providing a splint component which comprises a planar sheet of a material having an upper edge, a lower edge and two side edges which splint component can be readily rolled up from either of the side edges but resists being rolled up from either the top edge or the bottom edge, the splint component equipped with straps for retaining the splint component in a rolled up configuration;
orienting the splint component so that the side edges are parallel to a patient;
sliding the splint component beneath the patient so that the lower edge of the splint component is located in the vicinity of the patient's knees and the upper edge of the splint component is located in the vicinity of the patient's upper abdomen;
rolling up the two side edges over the patient to envelop the patent; and
connecting and tightening the straps whereby the hip and pelvic regions are immobilized by the splint component.

17. The method according to claim 16, further comprising the steps of providing a resilient foam insert sized to fit between the legs of the patient and placing the foam insert between the legs of the patient to further stabilize the legs when the patient is enveloped by the splint component.
